(19) United States
(12) Patent Application Publication
(10) Pub. No.: US 2021/0193326 A1
(43) Pub. Date: Jun. 24, 2021

(54) METHOD AND APPARATUS OF PROVIDING OSTEOARTHRITIS PREDICTION INFORMATION

(71) Applicant: ELECTRONICS AND TELECOMMUNICATIONS RESEARCH INSTITUTE, Daejeon (KR)

(72) Inventor: Hang-Kee KIM, Daejeon (KR)

(21) Appl. No.: 17/126,420

(22) Filed: Dec. 18, 2020

(30) Foreign Application Priority Data
Dec. 24, 2019 (KR) 10-2019-0174040

(51) Int. Cl. G16H 50/50; G16H 30/40; G16H 50/20; G16H 70/60; G06F 30/23

(52) U.S. Cl. CPC G16H 50/50; G16H 30/40; G06F 30/23; G16H 70/60; G16H 50/20

(57) ABSTRACT

A method for providing osteoarthritis prediction information includes acquiring input data including medical image data corresponding to an image of a joint area of a user, generating a joint model of the joint area by performing a simulation based on finite element analysis (FEA) for the input data, generating bone tissue pattern information for the joint area using the input data, predicting disease in the joint area using the joint model and the bone tissue pattern information, and providing the result of prediction of the disease in the joint area to the user.

### CROSS REFERENCE TO RELATED APPLICATION

This application claims the benefit of Korean Patent Application No. 10-2019-0174040, filed Dec. 24, 2019, which is hereby incorporated by reference in its entirety into this application.

### BACKGROUND OF THE INVENTION

### 1. Technical Field

The present invention relates generally to a method and apparatus for providing osteoarthritis prediction information, and more particularly to a method and apparatus for predicting the possibility that osteoarthritis will occur or the degree of exacerbation of osteoarthritis using medical image data and providing the prediction result.

### 2. Description of Related Art

Osteoarthritis is a disease causing inflammation and pain while damaging bones and ligaments constituting joints due to damage to cartilage protecting the joints or a degenerative change in the cartilage.

If it is possible to diagnose osteoarthritis at an early stage or predict the possibility that osteoarthritis will occur, treatment for slowing the progress of osteoarthritis or lowering the possibility of occurrence of osteoarthritis may be prescribed.

### DOCUMENTS OF RELATED ART

(Patent Document 1) Korean Patent Application Publication No. 10-2011-0101009, published on Sep. 15, 2011 and titled “Biomarker indicative of arthritis and diagnosis of arthritis using the same”.

### SUMMARY OF THE INVENTION

An object of the present invention is to provide a method and apparatus for predicting the possibility that osteoarthritis will occur or the degree of exacerbation of osteoarthritis using medical image data and providing the prediction result.

Another object of the present invention is to provide a method and apparatus for providing a more accurate prediction result by using a simulation based on finite element analysis and bone tissue pattern information together in order to predict osteoarthritis.

In order to accomplish the above objects, a method for providing osteoarthritis prediction information according to an embodiment of the present invention includes acquiring input data including medical image data corresponding to an image of a joint area of a user, performing a simulation based on finite element analysis (FEA) for the input data and thereby generating a joint model of the joint area, generating bone tissue pattern information of the joint area using the input data, predicting disease in the joint area using the joint model and the bone tissue pattern information, and providing a result of prediction of the disease in the joint area to the user.

Also, in order to accomplish the above objects, an apparatus for providing osteoarthritis prediction information according to an embodiment of the present invention includes memory in which at least one program is recorded and a processor for executing the program. The program includes instructions for acquiring input data including medical image data corresponding to an image of a joint area of a user, generating a joint model of the joint area by performing a simulation based on finite element analysis (FEA) for the input data, generating bone tissue pattern information of the joint area using the input data, predicting disease in the joint area using the joint model and the bone tissue pattern information, and providing a result of prediction of the disease in the joint area to the user.

### BRIEF DESCRIPTION OF THE DRAWINGS

The above and other objects, features and advantages of the present invention will be more clearly understood from the following detailed description, taken in conjunction with the accompanying drawings, in which:

FIG. 1 is a view illustrating a method for providing osteoarthritis prediction information according to an embodiment of the present invention;

FIG. 2 is a flowchart illustrating a method for providing osteoarthritis prediction information according to another embodiment of the present invention;

FIG. 3 is a view illustrating a method for providing osteoarthritis prediction information according to a further embodiment of the present invention;

FIG. 4 is a view illustrating an example of an input value for machine learning;

FIG. 5 is a view illustrating another example of an input value for machine learning;

FIG. 6 is a view illustrating an example of a disease prediction result;

FIG. 7 is a view illustrating another example of a disease prediction result; and

FIG. 8 is a view illustrating a computer system according to an embodiment of the present invention.

### DESCRIPTION OF THE PREFERRED EMBODIMENTS

The present invention will be described in detail below with reference to the accompanying drawings. Repeated descriptions and descriptions of known functions and configurations that have been deemed to unnecessarily obscure the gist of the present invention will be omitted below. The embodiments of the present invention are intended to fully describe the present invention to a person having ordinary knowledge in the art to which the present invention pertains. Accordingly, the shapes, sizes, etc. of components in the drawings may be exaggerated in order to make the description clearer.

Throughout this specification, the terms “comprises” and/or “comprising” and “includes” and/or “including” specify the presence of stated elements but do not preclude the presence or addition of one or more other elements unless otherwise specified.

Hereinafter, a preferred embodiment of the present invention will be described in detail with reference to the accompanying drawings.

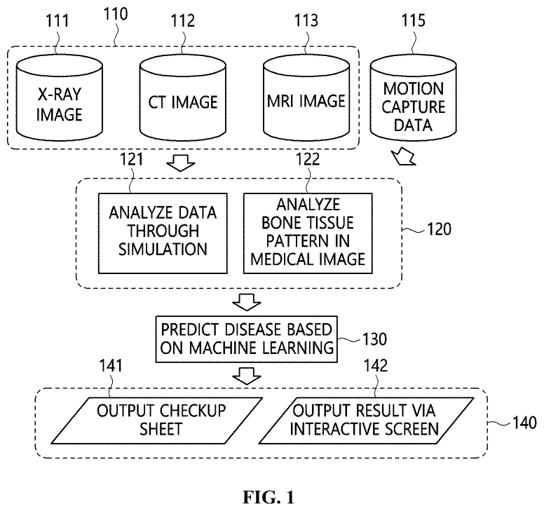

FIG. 1 is a view illustrating a method for providing osteoarthritis prediction information according to an embodiment of the present invention.

Referring to FIG. 1, the method for providing osteoarthritis prediction information according to an embodiment of the present invention includes collecting medical image data 110 and motion capture data 115, performing analysis (120) through simulation (121) and analysis of a bone tissue pattern (122) using the collected data, predicting joint disease using machine learning based on the analysis result (130), and printing a check-up sheet based on the predicted data (141) or outputting the same via an interactive screen (142) (140).

The types of medical image data to be used for analysis may include an X-ray image 111, a Computed Tomography (CT) image 112, a Magnetic Resonance Imaging (MRI) image 113, and the like. These images may be used together, or one type of imaging may be selected and used.

Here, an image of the motion (walking, running or the like) of a user is captured, and the motion capture data 115 is applied to the simulation, whereby the accuracy may be improved.

The motion capture data may include, for example, an angle of rotation of a joint, the load on the joint, and the like, which change over time.

When motion capture data of the user is not present, data about standard user motions (walking, running and the like) may be used for a simulation.

Based on such input data, a simulation based on 3D Finite Element Analysis (FEA) for a joint area and analysis of a bone tissue pattern may be performed.

The early signs of osteoarthritis are observed as degeneration of cartilage due to damage, a change in a bone tissue pattern, and the like. Accordingly, when an FEA-based simulation and analysis of a bone tissue pattern are performed, the possibility of a healthy person being afflicted with the disease in a few years may be predicted.

For example, based on the most commonly used Kellgren-Lawrence (KL) grade, the possibility that a person whose knee joints are currently determined to be KL 0 (normal) will be diagnosed as having knee joints graded as KL 2 in four years may be predicted as a percentage (%) value.

Bone tissue pattern analysis using medical image data uses the fact that a bone tissue pattern changes with the progression of osteoarthritis, and this change may be automatically identified based on the progress of the disease.

When the above-described FEA-based simulation data and bone tissue pattern data are provided as input values for machine learning (ML), the degree of severity of disease in a predetermined number of years (N) may be output therefrom.

Here, machine learning may fundamentally use a method in which a result is inferred through supervised learning using input-output pairs.

For example, a multi-layer perceptron, a support-vector machine, a random-forest algorithm, a deep-learning algorithm such as a convolutional neural network (CNN), and the like may be used, but the scope of the present invention is not limited thereto.

The output for explaining the result to the person involved based on the disease prediction result may be provided in two ways, that is, by printing a check-up sheet or by outputting the same via an interactive screen.

Printing a check-up sheet may be configured to print the test result on paper and provide the same to the user.

Outputting the test result via an interactive screen may be configured to provide the result using a device enabling interaction with a user, such as a user terminal or the like, such that the user is able to check individual items more dynamically and through a greater variety of methods.

Here, the user terminal indicates a communication terminal capable of using a terminal application in a wired/wireless communication environment. For example, the user terminal may be a personal computer or a portable terminal, such as a smartphone, but the spirit of the present invention is not limited thereto, and any terminal in which a terminal application is capable of being installed may be adopted without limitation.

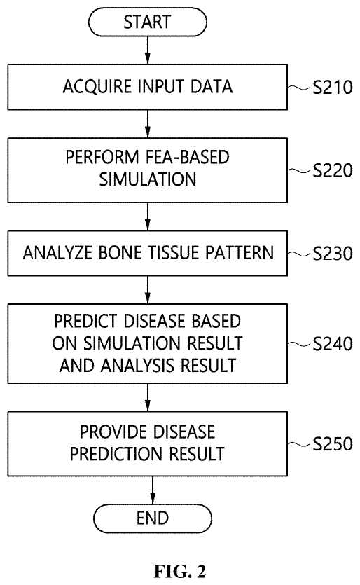

FIG. 2 is a flowchart illustrating a method for providing osteoarthritis prediction information according to another embodiment of the present invention.

Referring to FIG. 2, in the method for providing osteoarthritis prediction information according to another embodiment of the present invention, input data including medical image data corresponding to an image of a joint area of a user is acquired at step S210, a joint model for the joint area is generated at step S220 by performing a simulation based on Finite Element Analysis (FEA) for the input data, bone tissue pattern information for the joint area is generated using the input data at step S230, disease in the joint area is predicted using the joint model and the bone tissue pattern information at step S240, and the result of prediction of the disease in the joint area is provided to the user at step S250.

Here, the medical image data may be an image acquired using at least one of an X-ray, Computed Tomography (CT), and Magnetic Resonance Imaging (MRI) for the joint area.

Acquiring the input data at step S210 may be configured to acquire input data that further includes motion capture data corresponding to measurement data on the joint area.

Here, the motion capture data may be data acquired by measuring at least one of movement of a joint and a load on the joint over time when the joint area is moving.

Generating the joint model at step S220 may be configured to generate a 3D joint model for the joint area by performing a 3D-FEA-based simulation for the input data, to generate a joint cross-section image by performing 2D orthogonal projection on the 3D joint model, and to generate a joint model including the joint cross-section image.

Here, generating the joint cross-section image may be configured to set projection angles for respective bones included in the 3D joint model such that the longest axis of each of the bones and the projection plane are parallel to each other and to generate multiple joint cross-section images for the 3D joint model based on the projection angles.

That is, the joint model may include the multiple joint cross-section images.

Predicting the disease in the joint area at step S240 includes predicting a new joint model and a new bone tissue pattern after a preset time period based on the joint model and the bone tissue pattern information, and this may be a process of predicting the new joint model and the new bone tissue pattern after a preset time period based on a value output from a machine-learning model when the joint model and the bone tissue pattern information are input to the machine-learning model as input values thereof.

Here, the machine-learning model may be a model trained using a supervised learning method using joint models and bone tissue pattern information acquired from multiple users.

Predicting the disease in the joint area at step S240 may further include determining a Kellgren-Lawrence (KL) grade based on the joint model and the bone tissue pattern and predicting a KL grade after the preset time period.

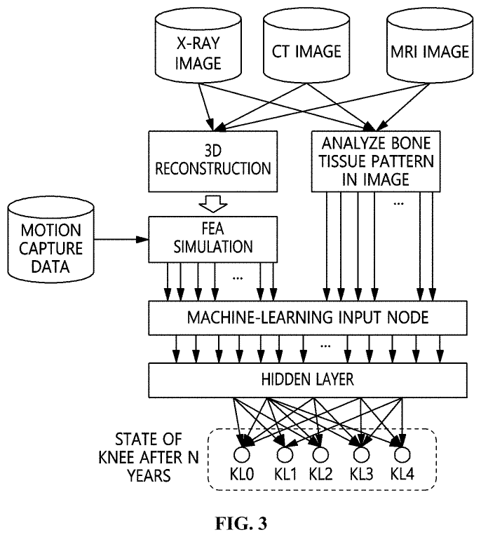

FIG. 3 is a view illustrating a method for providing osteoarthritis prediction information according to a further embodiment of the present invention.

Particularly, FIG. 3 illustrates a method for providing osteoarthritis prediction information by performing two types of analysis including FEA-based simulation and bone tissue pattern analysis and performing machine learning based on the analysis result.

Referring to FIG. 3, medical image data is used as the input of two analysis modules for an FEA-based simulation and bone tissue pattern analysis. In the case of the FEA-based simulation, a more accurate simulation may be performed by additionally receiving motion capture data of a user. When motion capture data of the user is not available, standard motion data may be used.

In order to perform the FEA-based simulation, first, 3D reconstruction of the medical image data may be performed. In the case of a CT or MRI image, any of various methods may be used in order to perform 3D reconstruction using information of each cross-section. In the case of an X-ray image, 3D reconstruction may be performed based on a smaller amount of information than when a CT or MRI image is used. In the case of biplanar X-rays, images acquired by scanning orthogonal front and side views are provided, the feature point or contour of the part desired to be reconstructed in 3D is specified and segmented, and Principal Component Analysis (PCA) parameters of a previously constructed statistical shape model are adjusted in order to find an optimal 3D shape, whereby 3D reconstruction may be performed.

The part to be reconstructed in 3D may be selected by considering the most important factor intended to be acquired as the result of a simulation. For example, in the case of a knee joint, the result of a simulation of the surface that comes into contact with femoral and tibial cartilage is the most important target, and reconstruction of a meniscus, a patella, or the like may be additionally performed for a more accurate simulation.

Based on the data reconstructed in 3D, an FEA-based simulation is performed. When the FEA-based simulation is performed, physical attributes of the 3D model may be set. For example, in the case of an elastic model, material information, such as mass, inertia tensor, Poisson's ratio, Young's modulus, and the like, and a gait load and a gait angle changing over time may be included in such physical attributes. Here, user-defined material may be configured for a more accurate cartilage simulation, in which case a more precise and complex simulation result may be acquired.

When the FEA-based simulation is performed as described above, a load on each contact area (e.g., stress, strain, and degeneration values) may be acquired as a result value.

Analysis of a bone tissue pattern in an image may be the process of finding a bone area in an entire medical image provided as input and performing image analysis on the tissue pattern of the bone area, thereby detecting the extent to which the bone area is changed. Finding a bone area in the image may be manually performed in such a way that a user specifies the bone area, or the bone area may be automatically found using an image-processing method (edge detection, contour detection, or the like) or machine learning. High-pass filtering through Fourier transform, Histogram Equalization (HE), normalization, or the like is performed as a preprocessing process for analyzing a bone tissue pattern, whereby identification of the bone tissue pattern may be facilitated.

In the above-mentioned machine-learning process, supervised learning may be performed as follows. When input data and correct output data corresponding thereto are regarded as Ground Truth (GT) and provided to input/output nodes, learning based on a learning algorithm is performed. Then, when new data is given as input, the inference result of the new data may be derived based on the learned content. Here, a hidden layer including at least one layer may be present between the input/output nodes.

In the present invention, data acquired by tracking at least one of an X-ray image, a CT image, an MRI image, and motion capture data of an existing patient over time, that is, time-series data (e.g., data of the base year, two years after that, four years after that, six years after that, and so on), may be used as Ground Truth (GT) data. Using the corresponding data as input, a 3D FEA-based simulation for each year and analysis of a bone tissue pattern for each year are performed, and the results thereof may be used as input for a machine-learning model. For the value corresponding to the output of the machine-learning model, the degree of severity of a symptom for each year (e.g., a KL grade) may be used.

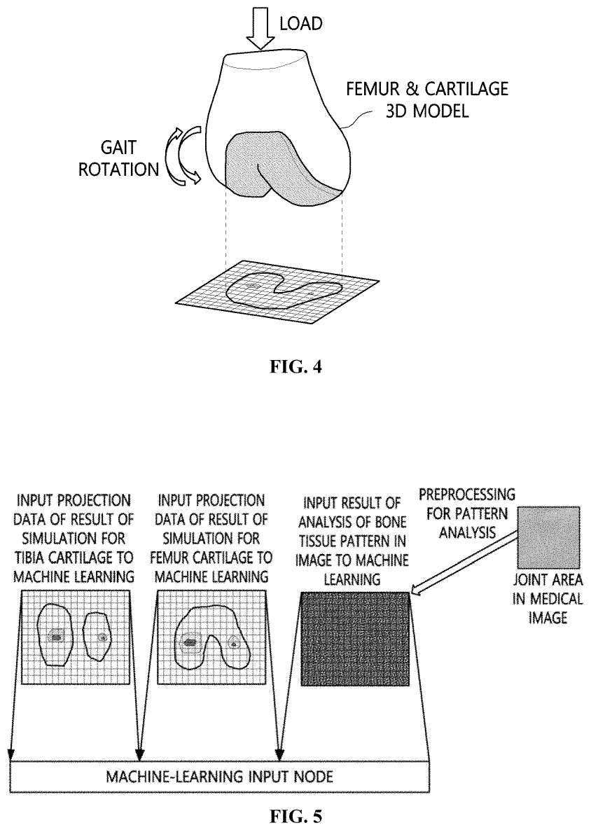

FIG. 4 is a view illustrating an example of an input value for machine learning.

Particularly, FIG. 4 is a view illustrating a method of generating an input value for machine learning by projecting the result of an FEA-based simulation.

Referring to FIG. 4, first, an apparatus for providing osteoarthritis prediction information according to an embodiment of the present invention may perform orthogonal projection for each of bones included in a 3D joint model, which is generated as the result of an FEA-based simulation, and may adjust the size of the orthogonal projection image so as to correspond to the input value of a machine-learning model.

Here, projection angles may be set such that the longest axis of each of the bones included in the 3D joint model is parallel to the plane of projection, and multiple joint cross-section images for the 3D joint model may be generated based on the projection angles.

In the case of a knee, projection data not only for femur cartilage but also for tibia cartilage, which comes into contact with a knee joint, may be generated and used as the input values of the machine-learning model. Further, a meniscus is also projected, and the projection data thereof may also be used as an input value of the machine-learning model.

FIG. 5 is a view illustrating another example of an input value for machine learning.

Particularly, FIG. 5 is a view illustrating a method of using the result values of an FEA-based simulation and bone tissue pattern analysis as the input values for a machine-learning model.

Referring to FIG. 5, an apparatus for providing osteoarthritis prediction information according to an embodiment of the present invention may use information acquired by orthogonally projecting the result of an FEA-based simulation and the result of preprocessing a bone tissue pattern as input values for a machine-learning model. The respective pieces of information may be delivered in a concatenated form. Further, additional data, such as the body mass index (BMI) of a user, the age of the user, and the like, may be used as the input values of the machine-learning model.

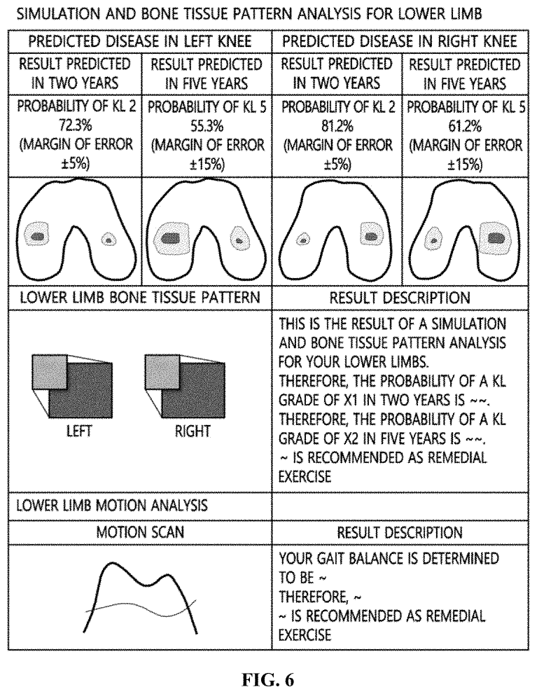

FIG. 6 is a view illustrating an example of a disease prediction result.

Referring to FIG. 6, the results of a simulation and bone tissue pattern analysis for lower limbs are represented as the probability that each disease will occur in each prediction year, and each simulation result, a pattern analysis result, and a lower limb movement analysis result may be provided in the form of images.

Also, in order to help a user understand the result, a description of the result may be provided in the form of a sentence. Here, the result description sentence may be provided by retrieving a sentence from a database, which stores sentences written in advance for respective results, or by combining or selecting sentences using the output value of the machine-learning model.

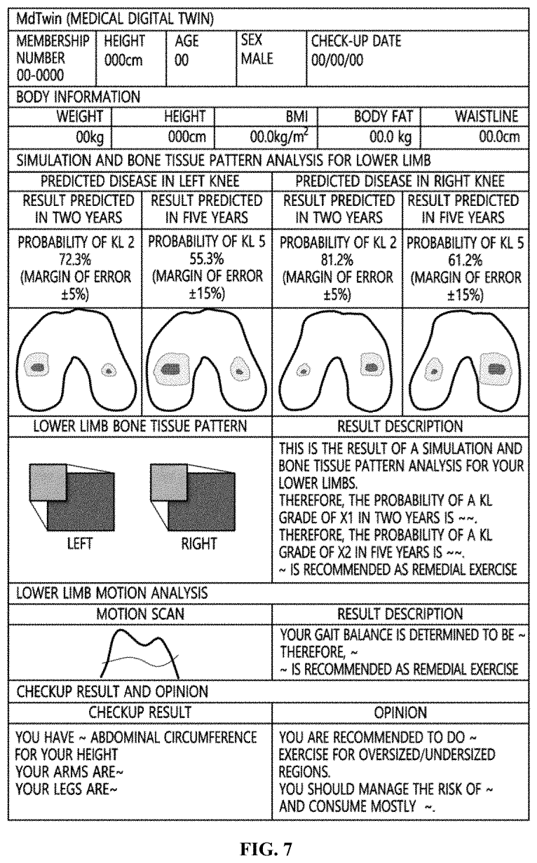

FIG. 7 is a view illustrating another example of a disease prediction result.

Particularly, FIG. 7 illustrates an example in which a disease prediction result in the form of a check-up sheet is provided to a user.

The check-up sheet is provided by printing a test result on paper, and may make it easy for a user to see the overall content at a glance and to carry the same. This check-up sheet is the result of a medical check-up result and takes a form that is easy for a user to receive, and may be provided along with a check-up sheet for another test.

Although not illustrated in FIG. 7, the disease prediction result may be provided to a user by outputting the same via an interactive screen. The interactive screen may be provided through a device such as a personal computer, a smart phone, or the like, and enables the user to check each item using a greater variety of methods.

For example, when a user clicks simulation data, animation of the simulation may be provided, and a change in the load on each part during the simulation may be provided as a change in the color of the part. Also, the progress of osteoarthritis can be observed in 3D from different angles. In the case of motion capture data, a skeleton, cartilage, muscle and the like constructed in 3D based on the medical images of the user may be visualized in the form of an animation and may then be provided.

FIG. 8 is a view illustrating a computer system according to an embodiment of the present invention.

The apparatus for providing osteoarthritis prediction information according to the present invention may be implemented as a computer system 600.

Referring to FIG. 8, the computer system 600 may include one or more processors 610, memory 630, a user-interface input device 640, a user-interface output device 650, and storage 660, which communicate with each other via a bus 620. Also, the computer system 600 may further include a network interface 670 connected with a network 680. The processor 610 may be a central processing unit or a semiconductor device for executing processing instructions stored in the memory 630 or the storage 660. The memory 630 and the storage 660 may be any of various types of volatile or nonvolatile storage media. For example, the memory may include ROM 631 or RAM 632.

Although specific embodiments have been described in the specification, they are not intended to limit the scope of the present invention. For the conciseness of the specification, descriptions of conventional electronic components, control systems, software, and other functional aspects thereof may be omitted. Also, lines connecting components or connecting members illustrated in the drawings show functional connections and/or physical or circuit connections, and may be represented as various functional connections, physical connections, or circuit connections capable of replacing or being added to an actual device. Also, unless specific terms, such as “essential”, “important”, or the like, are used, corresponding components may not be absolutely necessary.

According to the present invention, a method and apparatus for predicting the possibility that osteoarthritis will occur or the degree of exacerbation of osteoarthritis using medical image data and for providing the prediction result may be provided.

Also, according to the present invention, a simulation based on finite element analysis and bone tissue pattern information are used together in order to predict osteoarthritis, whereby a method and apparatus for providing a more accurate prediction result may be provided.

Accordingly, the spirit of the present invention should not be construed as being limited to the above-described embodiments, and the entire scope of the appended claims and their equivalents should be understood as defining the scope and spirit of the present invention.

What is claimed is:

1. A method for providing osteoarthritis prediction information, comprising:
acquiring input data including medical image data corresponding to an image of a joint area of a user;
performing a simulation based on finite element analysis (FEA) for the input data and thereby generating a joint model of the joint area;
generating bone tissue pattern information of the joint area using the input data;
predicting disease in the joint area using the joint model and the bone tissue pattern information; and
providing a result of prediction of the disease in the joint area to the user.

2. The method of claim 1, wherein generating the joint model comprises:
generating a 3D joint model of the joint area by performing a simulation based on 3D FEA for the input data;
generating a joint cross-section image by performing 2D orthogonal projection on the 3D joint model; and
generating the joint model including the joint cross-section image.

3. The method of claim 2, wherein:
generating the joint cross-section image is configured to set projection angles for respective bones included in the 3D joint model such that a longest axis of each of the bones is parallel to a plane of projection and to generate multiple joint cross-section images for the 3D joint model based on the projection angles, and
the joint model includes the multiple joint cross-section images.

4. The method of claim 1, wherein predicting the disease in the joint area comprises:
predicting a new joint model and a new bone tissue pattern after a preset time period based on the joint model and the bone tissue pattern information.

5. The method of claim 4, wherein predicting the new joint model and the new bone tissue pattern after the preset time period is configured to predict the new joint model and the new bone tissue pattern after the preset time period based on a value that is output from a machine-learning model when the joint model and the bone tissue pattern information are input to the machine-learning model as input values thereof.

6. The method of claim 5, wherein the machine-learning model is a model trained using a supervised learning method using joint models and bone tissue pattern information acquired from multiple users.

7. The method of claim 4, wherein predicting the disease in the joint area further comprises:
determining a Kellgren-Lawrence grade based on the joint model and the bone tissue pattern information and predicting a Kellgren-Lawrence grade after the preset time period.

8. The method of claim 1, wherein the medical image data corresponds to an image acquired using at least one of an X-ray, Computed Tomography (CT), and Magnetic Resonance Imaging (MRI) for the joint area.

9. The method of claim 1, wherein acquiring the input data is configured to acquire the input data that further includes motion capture data corresponding to measurement data on the joint area.

10. The method of claim 9, wherein the motion capture data is data acquired by measuring at least one of movement of a joint and a load on the joint over time when the joint area is moving.

11. An apparatus for providing osteoarthritis prediction information, comprising:
memory in which at least one program is recorded; and
a processor for executing the program,
wherein the program includes instructions for
acquiring input data including medical image data corresponding to an image of a joint area of a user,
generating a joint model of the joint area by performing a simulation based on finite element analysis (FEA) for the input data,
generating bone tissue pattern information of the joint area using the input data,
predicting disease in the joint area using the joint model and the bone tissue pattern information, and
providing a result of prediction of the disease in the joint area to the user.

12. The apparatus of claim 11, wherein generating the joint model comprises:
generating a 3D joint model of the joint area by performing a simulation based on 3D FEA for the input data;
generating a joint cross-section image by performing 2D orthogonal projection on the 3D joint model; and
generating the joint model including the joint cross-section image.

13. The apparatus of claim 12, wherein:
generating the joint cross-section image is configured to set projection angles for respective bones included in the 3D joint model such that a longest axis of each of the bones is parallel to a plane of projection and to generate multiple joint cross-section images for the 3D joint model based on the projection angles, and
the joint model includes the multiple joint cross-section images.

14. The apparatus of claim 11, wherein predicting the disease in the joint area comprises:
predicting a new joint model and a new bone tissue pattern after a preset time period based on the joint model and the bone tissue pattern information.

15. The apparatus of claim 14, wherein predicting the new joint model and the new bone tissue pattern after the preset time period is configured to predict the new joint model and the new bone tissue pattern after the preset time period based on a value that is output from a machine-learning model when the joint model and the bone tissue pattern information are input to the machine-learning model as input values thereof.

16. The apparatus of claim 15, wherein the machine-learning model is a model trained using a supervised learning method using joint models and bone tissue pattern information acquired from multiple users.

17. The apparatus of claim 14, wherein predicting the disease in the joint area further comprises:
determining a Kellgren-Lawrence grade based on the joint model and the bone tissue pattern information and predicting a Kellgren-Lawrence grade after the preset time period.

18. The apparatus of claim 11, wherein the medical image data corresponds to an image acquired using at least one of an X-ray, Computed Tomography (CT), and Magnetic Resonance Imaging (MRI) for the joint area.

19. The apparatus of claim 11, wherein acquiring the input data is configured to acquire the input data that further includes motion capture data corresponding to measurement data on the joint area.

20. The apparatus of claim 19, wherein the motion capture data is data acquired by measuring at least one of movement of a joint and a load on the joint over time when the joint area is moving.
